(19) United States
(12) Patent Application Publication
(10) Pub. No.: US 2008/0200557 A1
(43) Pub. Date: Aug. 21, 2008

(54) Method for Inhibiting Lipid Peroxidation

(75) Inventors: Gregory Okenkrug, Newton, MA (US); Pura J. Requintina, West Kingston, RI (US)

Correspondence Address: NIXON PEABODY, LLP, 401 9TH STREET, NW, SUITE 900, WASHINGTON, DC, 20004-2128, US

(73) Assignee: CARITAS ST. ELIZABETH HOSPITAL OF BOSTON, INC., Boston, MA (US)

(21) Appl. No.: 11/630,888

(22) PCT Filed: Jul. 7, 2005
(86) PCT No.: PCT/US2005/024023

§ 371 Date: Dec. 19, 2007

Related U.S. Application Data
(60) Provisional application No. 60/585,902, filed on Jul. 7, 2004.

(51) Int. Cl. A61K 31/135; A61P 31/00; A61P 25/00; A61P 17/00

(52) U.S. Cl. 514/653

(57) ABSTRACT

The invention relates generally to the use of N-substituted dopamine derivatives for the treatment of diseases and disorders that involve abnormal lipid peroxidation. This method comprises the administration of a pharmaceutically effective amount of N-acetyldopamine derivatives or N-alkyldopamine derivatives and a pharmaceutically acceptable carrier for treating an animal or human suffering abnormal lipid peroxidation. The N-acetyldopamine derivative or N-alkyldopamine derivatives may be administered alone or in combination with N-acetylserotonin (NAS) to inhibit lipid peroxidation.

### FIELD OF THE INVENTION

The invention relates generally to a method of treating abnormal lipid peroxidation associated with a disease or disorder. In particular, N-substituted dopamine derivatives may be used alone or in combination with N-acetylserotonin (NAS) in the treatment of diseases and disorders that are associated with lipid peroxidation.

### BACKGROUND OF THE INVENTION

Septic shock represents a major cause of death in intensive care units. Septic shock is characterized by hypotension, hyporeactivity to vasoconstrictor agents, inadequate tissue perfusion, vascular damage and disseminated intravascular coagulation leading to multiple organ failure and death. Systemic inflammatory response syndrome (SIRS) is the response of the body to severe trauma or non-infectious disorders (Tracey and Cerami, 1994; Reynolds et al., 2003). SIRS has the same pathogenic mechanisms and clinical presentation as septic shock but no identifiable infection focus.

Pathogenic mechanisms of septic shock involve the release of bacterial Gram-negative membrane component, lipopolysaccharide (LPS), which triggers the production of the inflammatory cytokines such as tumor necrosis factor-alpha (TNF-α), and of inducible nitric oxide synthase (iNOS), and results in extensive oxidative damage with the formation of reactive oxygen species (see Maestroni, 1996). Treatment of septic shock is directed primarily toward establishing a specific microbial diagnosis and an entry site for the responsible microorganisms. Therapy involves use of antibiotics, glucocorticoids and necessary surgical intervention to drain abscesses or to remove infected foreign bodies.

Oxidation is a part of the normal metabolism of the human body. In these metabolic processes highly reactive molecules, free radicals, are processed. Free radicals are atoms or molecules that contain one or more unpaired electrons. These compounds have important physiologic functions e.g. for the function of phagocytes, but they are also capable to induce oxidative damage to essential biomolecules such as nucleic acids, lipids and proteins. Lipid peroxidation can be defined as the oxidative deterioration of lipids containing any number of carbon-carbon double bonds. Abnormal lipid peroxidation is a central feature of oxidative damage, which plays a key role in LPS-induced septic shock. Melatonin, the pineal hormone has been shown to counteract the toxic effect of LPS in laboratory animals (Sewerynek et al., 1995; Maestroni, 1996; Sacco et al., 1998; Requintina and Oxenkrug, 2003) and prevent lethality in patients suffering from septic shock (Gitto et al., 2004). N-acetylserotonin (NAS), the immediate precursor and metabolite (Young et al., 1985) of melatonin, protects rats against LPS-induced hypotensive shock (Klemm et al., 1993) and LPS lethal effect in mice (Requintina et al., 2003).

U.S. Pat. No. 5,578,570 discloses a method of treating septic shock in a mammal including the administration of a septic shock-treating effective amount of thymosin β₄ by obstructing the progression of sepsis cascade. U.S. Pat. No. 5,627,173 discloses that Phosphonoacetic acid derivatives and their use for treating degenerative joint disorders of rheumatic disorders accompanied by cartilage breakdown, such as rheumatoid arthritis, joint trauma and chondrolysis as a consequence of prolonged immobilization of the joint, of inflammations, septic shock, disorders with impaired leukocyte adhesion, disorders caused by an elevated concentration of tumor necrosis factor alpha, such as cachexia or Crohn's disease.

U.S. Pat. No. 5,726,156 discloses novel peptides that are potent cytokine regulatory agents to treat such conditions including diseases mediated by nitric oxide and cytokines, adverse drug reactions, obesity, septic shock, and adverse side effects due to cancer chemotherapy or occurring as in response to organ transplantation. U.S. Pat. No. 5,939,394 discloses that magnesium gluconate has been developed as compositions for intervention with to inhibit formation of lipid peroxidation products and cytokine-mediated abnormalities to prevent and/or treat immunological disorders, inflammatory diseases and chronic infections.

Despite the prior work on treating septic shock, additional methods of inhibiting lipid peroxidation are needed. This invention answers that need.

### BRIEF DESCRIPTION OF THE DRAWINGS

FIG. 1 depicts the effect of different concentrations of N-acetyldopamine on the level of lipid peroxidation induced by lipopolysaccharide (LPS) in rat brain tissues.

FIG. 2 depicts the effect of different concentrations of N-acetylserotonin on the level of lipid peroxidation induced by lipopolysaccharide (LPS) in rat brain tissues.

FIG. 3 shows the effect of different concentrations of melatonin on the level of lipid peroxidation induced by lipopolysaccharide (LPS) in rat brain tissues.

FIG. 4 shows the effect of combination of NAS and N-acetyldopamine on the level of lipid peroxidation induced by lipopolysaccharide (LPS) in rat brain tissues.

FIG. 5 shows the effect of combination of melatonin and N-acetyldopamine on the level of lipid peroxidation induced by lipopolysaccharide (LPS) in rat brain tissues.

FIG. 6 shows the effect of different concentrations of 3-methoxydopamine (4-hydroxyl-3-methoxy phenethylamine) on the level of lipid peroxidation induced by lipopolysaccharide (LPS) in mice brain tissues.

FIG. 7 shows the effect of different concentrations of 4-methoxydopamine (3-hydroxy-4-methoxy phenethylamine) on the level of lipid peroxidation induced by lipopolysaccharide (LPS) in mice brain tissues.

FIG. 8 shows that the effect of different concentrations of N-acetylserotonin on lipid peroxidation induced by FeCl₂ in mice brain tissues.

FIG. 9 shows that the effect of different concentrations of N-acetyldopamine on lipid peroxidation induced by FeCl₂ in mice brain tissues.

FIG. 10 shows that the effect of different concentrations of N-methyldopamine (deoxyepinephrine) on lipid peroxidation induced by FeCl₂ in mice brain tissues.

FIG. 11 shows that the effect of different concentrations of 4-methoxydopamine (3-hydroxy-4-methoxy phenethylamine) on lipid peroxidation induced by FeCl₂ in mice brain tissues.

FIG. 12 shows that the effect of different concentrations of 3-methoxydopamine (4-hydroxy-3-methoxy phenethylamine HCl) on lipid peroxidation induced by FeCl₂ in mice brain tissues.

### SUMMARY OF THE INVENTION

The invention relates to a method for treating an animal or human suffering abnormal lipid peroxidation associated with a disease or disorder. This method comprises the administration of a pharmaceutically effective amount of N-substituted dopamine derivatives and a pharmaceutically acceptable carrier for treating an animal or human suffering abnormal lipid peroxidation. N-substituted dopamine derivatives may be N-acetyldopamine derivatives or N-alkyldopamine derivatives. N-substituted dopamine derivatives may be administered alone or in combination with a pharmaceutically effective amount of N-acetylserotonin (NAS) to prevent or inhibit lipid peroxidation.

According to the invention, N-substituted dopamine derivatives exhibit beneficial therapeutic properties and are useful in the treatment of lipid peroxidation associated with a disease or disorder. The disease or disorder may be septic shock. Alternatively, the disease or disorder may be a systemic inflammatory response syndrome (SIRS), microbial infection, carcinogenesis, a mutation, or a degenerative disorder, such as, for example, Parkinson's or Alzheimer's disease. Alternatively, the disease or disorder may be multiple sclerosis, a burn, aging, metabolic syndrome (dyslipidemia, pre- and diabetes, hypertention) or the toxic effects of chemotherapy or radiation therapy.

### DETAILED DESCRIPTION OF THE INVENTION

The invention relates to a method for treating an animal or human suffering abnormal lipid peroxidation associated with a disease or disorder. This method comprises the administration of a pharmaceutically effective amount of N-substituted dopamine derivatives and a pharmaceutically acceptable carrier for treating an animal or human suffering abnormal lipid peroxidation. The invention further provides a method of for treating an animal or human suffering abnormal lipid peroxidation comprising: administering an effective amount of N-substituted dopamine derivatives in combination with an effective amount of N-acetylserotonin.

The pharmaceutical composition of the invention may be employed in the treatment of any condition associated with abnormal lipid peroxidation such as endothelial dysfunction, aging (including senescence-associated changes in skin and appearance) and diseases like diabetes mellitus, cardiovascular diseases (such as ischaemic heart disease, angina pectoris, myocardial infraction, congestive heart failure, atherosclerosis, hypertension and arrhythmia), obesity, asthma, trauma, shock (hypovolumic, neurogenic or septic), neurotoxicity, neurodegenerative and neurological disorders (including Alzheimer and Parkinson's diseases, amyotrophic lateral sclerosis, multiple sclerosis, convulsive (seizure) disorders, AIDS-dementia and disorders which involve processes of teaming and memory), disorders of gastric secretions, relaxation and peristalsis of the intestinal tract (including inflammatory bowel diseases), drug and disease-induced nephropathies, pathological (premature) and physiological uterine contractions, cellular defense impairment, endothelial dysfunction-induced diseases and insulin-resistance in diabetes, pregnancy-induced hypertension, chemotaxis and phagocytic impairment in immunological disorders, cerebrovascular diseases, aggregation disorders, fertility and reproductive disorders (erg, penile erection and treatment of male impotence). Abnormal lipid peroxidation is excessive lipid peroxidation often associated with a disease or disorder such as those discussed above.

N-substituted dopamine derivatives useful in the invention includes N-acetyldopamine derivatives or N-alkyldopamine derivatives. The N-substituted dopamine derivatives include compounds of Formula (I):

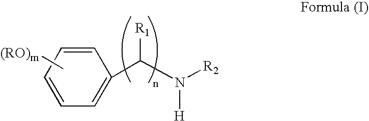

or a pharmaceutically acceptable salt or prodrug form thereof,
wherein
R is independently hydrogen, optionally substituted C₁-C₆ alkyl, optionally substituted C₂-C₆ alkenyl, and optionally substituted C₂-C₆ alkynyl;
m is 0, 1, 2, 3, 4 or 5;
R₁ is independently hydrogen, halogen, optionally substituted C₁-C₆ alkyl, optionally substituted C₂-C₆ alkenyl, optionally substituted C₂-C₆ alkynyl, optionally substituted C₁-C₆ alkoxy, optionally substituted C₁-C₆ alkylthio, optionally substituted C₁-C₆ alkylsulfinyl, optionally substituted C₁-C₆ alkylsulfonyl, optionally substituted C₁-C₆ aminoalkyl, optionally substituted carbocyclic aryl, or optionally substituted aralkyl;
n is 1, 2, or 3;
R₂ is optionally substituted C₁-C₆ alkyl or —C(═O)R₃;
R₃ is independently optionally substituted C₁-C₆ alkyl, optionally substituted C₂-C₆ alkenyl, optionally substituted C₂-C₆ alkynyl, optionally substituted or unsubstituted carbocyclic aryl, or an optionally substituted heteroaromatic or heteroalicyclic group having from 1 to 3 rings, 3 to about 8 ring members in each ring and 1 to about 3 hetero atoms.

As indicated in Formula (I), for the N-substituted dopamine derivatives used in the invention, R₂ is optionally substituted C₁-C₆ alkyl, or —C(═O)R₃. In the preferred compounds, R₂ is —CH₃ or —C(═O)R₃.

The term “alkyl,” unless otherwise modified, refers to straight chain and branched alkyls as well as cyclic groups, although of course cyclic groups will comprise at least three carbon ring members.

Preferred alkyl groups of compounds of Formula (I) have from one to 6 carbon atom, more preferably one to six atoms, and even more preferably 1, 2, or 3 atoms. The alkenyl and alkynyl groups of compounds of the invention have one or more unsaturated linkages and typically from 2 to about 6 carbon atoms, and more preferably 2 to about 4 carbon atoms. The terms “alkenyl” and “alkynyl” as used herein refer to both straight chain or branched cyclic groups and noncyclic groups, although straight or branched noncyclic groups are generally more preferred.

The term “substituted” mean that the substitution with common substituents known in the art. Examples of such substituents include hydroxyl groups, thiol groups, halogens, amino groups, nitro groups, sulphate groups, phosphate groups, carboxylic acid groups, esters, amides, and the like.

Preferred alkoxy groups of compounds of the invention include groups having one or more oxygen linkages and from 1 to about 6 carbon atoms, and more preferably from 1 to about 4 carbon atoms. The alkoxy groups may be straight chain or branched groups, saturated or unsaturated.

Preferred alkylthio groups of compounds of the invention include those groups having one or more thioether linkages and from 1 to about 6 carbon atoms, and more preferably from 1 to about 4 carbon atoms. The alkylthio groups may be straight chain or branched groups, saturated or unsaturated as well as cyclic groups, although of course cyclic groups will comprise at least three carbon ring members.

Preferred alkylsulfinyl groups of compounds of the invention include those groups having one or more sulfoxide (SO) groups and from 1 to about 6 carbon atoms, and more preferably from 1 to about 4 carbon atoms. The alkylsulfinyl groups may be straight chain or branched groups, saturated or unsaturated as well as cyclic groups, although of course cyclic groups will comprise at least three carbon ring members.

Preferred alkylsulfonyl groups of compounds of the invention include those groups having one or more sulfonyl (SO₂) groups and from 1 to about 6 carbon atoms, and more preferably from 1 to about 4 carbon atoms. The alkylsulfonyl groups may be straight chain or branched groups, saturated or unsaturated as well as cyclic groups, although of course cyclic groups will comprise at least three carbon ring members.

Preferred aminoalkyl groups include those groups having one or more primary, secondary and/or tertiary amine groups, and from 1 to about 6 carbon atoms, and more preferably 1 to about 4 carbon atoms. Secondary and tertiary amine groups are generally more preferred than primary amine moieties. The aminoalkyl groups may be straight chain or branched groups, saturated or unsaturated as well as cyclic groups, although of course cyclic groups will comprise at least three carbon ring members.

It should be understood that alkoxy, alkylthio, alkylsulfinyl, alkylsulfonyl and aminoalkyl substituent groups described above include groups where a hetero atom is directly bonded to a ring system, such as a carbocyclic aryl group or a heterocyclic group, as well as groups where a hetero atom of the group is spaced from such ring system by an alkylene linkage, e.g. of 1 to about 4 carbon atoms.

Without wishing to be bound by theory, compounds of the invention that contain an alkylsulfinyl and/or alkylsulfonyl group, may be, in effect, “pro-drugs” wherein after administration of the compound to a subject the sulfinyl or sulfonyl group(s) are metabolized (reduced) in vivo to the corresponding sulfide moiety.

As is known in the art, pharmaceutically acceptable salts of the N-substituted dopamine derivatives may also be used in the methods of invention. Suitable pharmaceutically acceptable salts include acid addition salts such as those prepared from the following acids: hydrochloric, hydrobromic, hydroiodic, sulfuric, nitric, phosphoric, maleic, salicylic, p-toluenesulfonic, tartaric, citric, acetic, methanesulfonic, formic, succinic, naphthalene-2-sulfonic, isethionic, lactobionic and benzenesulfonic.

Suitable heteroaromatic groups of compounds of the invention contain one or more N, O or S atoms and include, e.g., coumarinyl including 8-coumarinyl, quinolinyl including 8-quinolinyl, pyridyl, pyrazinyl, pyrimidyl, furyl, pyrrolyl, thienyl, thiazolyl, oxazolyl, oxidizolyl, triazole, imidazolyl, indolyl, benzofuranyl and benzothiazol.

Suitable hetefoalicyclic groups of compounds of the invention contain one or more N, O or S atoms and include, e.g., tetrahydrofuranyl, thienyl, tetrahydropyranyl, piperidinyl, morpholino and pyrrolindinyl groups.

Suitable carbocyclic aryl groups of compounds of the invention include single and multiple ring compounds, including multiple ring compounds that contain separate and/or fused aryl groups. Typical carbocyclic aryl groups of compounds of the invention contain 1 to 3 separate or fused rings and from 6 to about 18 carbon ring atoms. Specifically preferred carbocyclic aryl groups include phenyl; naphthyl including 1-naphthyl and 2-naphthyl; biphenyl; phenanthryl; anthracyl; and acenaphthyl. Substituted carbocyclic groups are particularly suitable including substituted phenyl, such as 2-substituted phenyl, 3-substituted phenyl, 4-substituted phenyl, 2,3-substituted phenyl, 2,5-substituted phenyl, 2,3,5-substituted and 2,4,5-substituted phenyl; and substituted naphthyl, including naphthyl substituted at the 5, 6 and/or 7 positions. Preferred substituents of such substituted carbocyclic groups are identified below.

Suitable aralkyl groups of compounds of the invention include single and multiple ring compounds, including multiple ring compounds that contain separate and/or fused aryl groups. Typical aralkyl groups contain alkyl chain as discussed above and 1 to 3 separate or fused rings and from 6 to about 18 carbon ring atoms. Preferred aralkyl groups include benzyl and methylenenaphthyl (—CH₂-naphthyl).

Specifically preferred compounds of the invention include N-acetyldopamine, N-chloroacetyldopamine, N-methyldopamine (deoxyepinephrine) or N-acetyl-m-tyramine to treat lipid peroxidation, according to the invention. N-acetyldopamine, N-chloroacetyldopamine, N-methyldopamine (deoxyepinephrime), N-acetyl-m-tyramine, and NAS may be prepared by those methods known in the art or purchased commercially.

It will be appreciated that the actual preferred amounts of active compounds used in a given therapy will vary according to the specific compound being utilized, the particular compositions formulated, the mode of application, the particular site of administration, etc. Optimal administration rates for a given protocol of administration can be readily ascertained by those skilled in the art using conventional dosage determination tests conducted with regard to the foregoing guidelines. The precise dosage will naturally depend on a number of clinical factors, for example, the age of the recipient, the route of administration and the condition under treatment, its severity, and whether one or a combination of compounds is administered. The desired daily dose is preferably given as two or three or more subdoses administered at appropriate intervals during the day.

According to the methods of the invention, the compounds of Formula (I) may be administered for the treatment of each of the disorders stated herein above, in the dosage range of 0.01 mg/kg to 500 mg/kg of human body weight per day, preferably about 0.1 mg/kg to about 50 mg/kg of human body weight per day and optimally about 10 mg/kg of human body weight per day.

Suitable effective dose of N-acetylserotonin in the combination of the compound of Formula (I) will be in the range of from 0.01 to 100 milligrams per kilogram of bodyweight of recipient per day, preferably in the range of from 0.01 to 20 milligrams per kilogram bodyweight of recipient per day, more preferably in the range of 0.05 to 4 milligrams per kilogram bodyweight of recipient per day.

While it is possible to administer N-acetyldopamine, N-chloroacetyldopamine, N-acetyl-m-tyramine, or NAS as a raw chemical, it is highly desirable to administer it in the form of a pharmaceutical composition comprising N-acetyldopamine, N-chloroacetyldopamine, N-acetyl-m-tyramine, or NAS together with an acceptable carrier therefor; the carrier should be acceptable in the sense of being compatible with the other ingredients and not deleterious to the recipient thereof. The compositions may be adapted for oral, transdermal, parenteral or rectal administration. Oral administration is preferred. In case of septic shock and SIRS the parenteral administration is preferred.

The pharmaceutical compositions may conveniently be presented in unit dosage form and may be prepared by any methods well known in the art of pharmacy. Preferred unit dosage formulations are those containing a daily subdose, as herein above recited, or an appropriate fraction thereof, of the active ingredient. Such methods include the step of bringing into association the active ingredient with the carrier which may comprise one or more accessory ingredients. In general the pharmaceutical compositions are prepared by uniformly and intimately bringing into association the active ingredient with liquid carriers or finely divided solid carriers or both, and then, if necessary, shaping or encapsulating the product.

Pharmaceutical compositions suitable for oral administration may be presented in discrete units such as capsules, cachets or tablets each containing a predetermined amount of active ingredient; as a powder or granules; as a solution or a suspension in an aqueous liquid or a non-aqueous liquid; or as an oil-in-water liquid emulsion or a water-in-oil liquid emulsion.

A tablet may be made by compression or molding, optionally with one or more accessory ingredients. Compressed tablets may be prepared by compressing in a suitable machine the active ingredient in a free-flowing form such as a powder of granules, optionally mixed with a binder, lubricant, inert diluent, and surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein.

The pharmaceutical compositions of the invention may be formulated into other forms depending upon the desired route of administration as is know in the art. Pharmaceutical compositions suitable for rectal administration may be presented as a suppository with the usual carriers such as cocoa butter. Pharmaceuticals compositions suitable for parenteral administration include aqueous sterile injection solutions which may contain antioxidants, buffers, bacteriostats and solutes which renders the parenteral composition isotonic with the blood of the intended recipient; and aqueous and non-aqueous sterile suspensions which may include suspending agents and thickening agents. The parenteral compositions may be presented in unit doses or multidose containers, for example, sealed ampoules and vials, and may be stored in a freeze dried (lyophilized) condition requiring only the addition of the sterile liquid carrier, for example, PEG 400: ethanol mixtures, immediately prior to use. Extemporaneous injection solutions and suspensions may be prepared from sterile powders, granules and tablets of the kind previously described.

It should be understood that in addition to the ingredients particularly mentioned above, the pharmaceutical compositions of this invention may include other agents conventional in the art having regard to the type of composition in question, for example, those suitable for oral administration may include flavoring agents.

According to the invention the N-substituted dopamine derivatives may be administered alone or in combination with other therapeutically effective agents. Use of N-substituted dopamine derivatives in combination with other drugs used presently in inhibiting lipid peroxidation such as, for example, NAS, may reduce the concentration of drug needed for successful treatment, thereby alleviating some of the major side effects observed with higher doses. Furthermore, the period observed between administering the drugs and any observed therapeutic indications may be diminished.

### EXAMPLES

### Example 1

The effect of N-acetyldopamine or the combination of N-acetyldopamine and NAS or the combination of N-acetyldopamine and melatonin on LPS-induced oxidative damage was evaluated in an in-vitro model. The effect of N-acetyldopamine on lipid peroxidation was compared to N-acetylserotonin (NAS) and melatonin, a powerful endogenous antioxidant (Reiter et al., 2002).

N-acetyldopamine, melatonin, 2-thiobarbituric acid, and LPS (Escherichia coli 0127:B8) were purchased from Sigma Chemical Co. (St. Louis, Mo.). The formation of thiobarbituric acid reactive substances (TBARS) was used as index of induced oxidative damage to lipid membranes. Lipid peroxidation was induced with LPS (400 μg/ml).

TBARS Determination

Brain tissues from Fisher 344N male rats were homogenized in 20 mM pH 7.4 Tris buffer. The homogenates were then incubated (1 hr) in solvent alone (control) or in solvent+LPS (400 μg/ml), N-acetyldopamine+LPS, NAS+LPS, melatonin+LPS, and N-acetyldopamine+NAS+LPS (for concentrations of N-acetyldopamine, NAS and melatonin, see Results). TBARS were added, and homogenates were incubated for 20 min. in a boiling water bath. After cooling, the absorbance was measured spectrophotometrically at 532 nm. TBARS concentrations were derived from a standard curve of MDA (Oxis International, Portland, Oreg.). Protein was determined by Lowry method (Sigma Chem. Co., St. Louis, Mo.). Results are expressed as mean+S.D. (n=6). Data was treated by one-way ANOVA and Student's t-test.

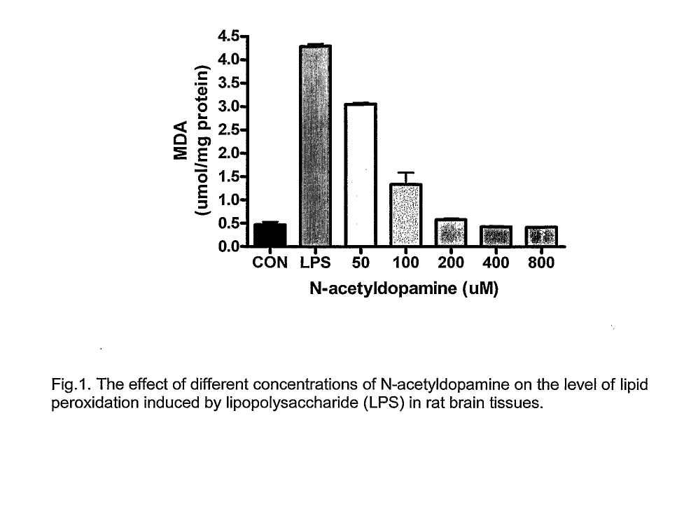

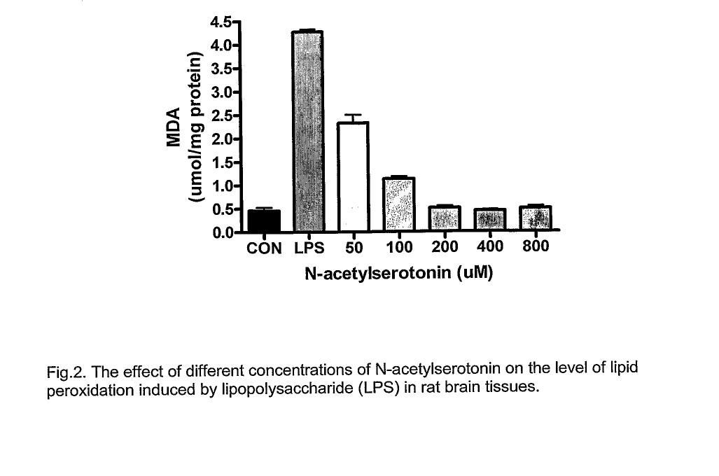

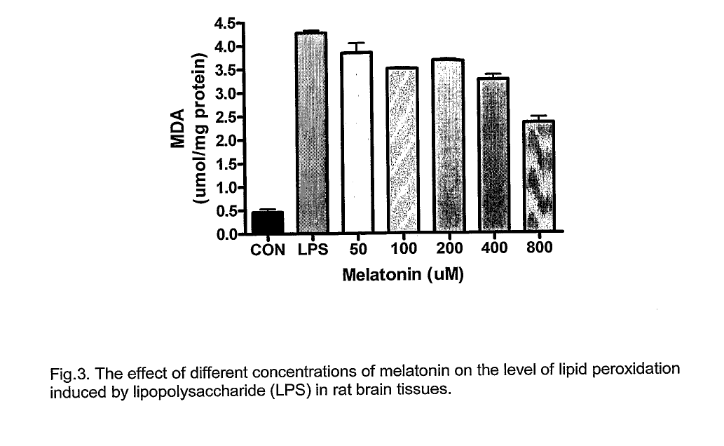

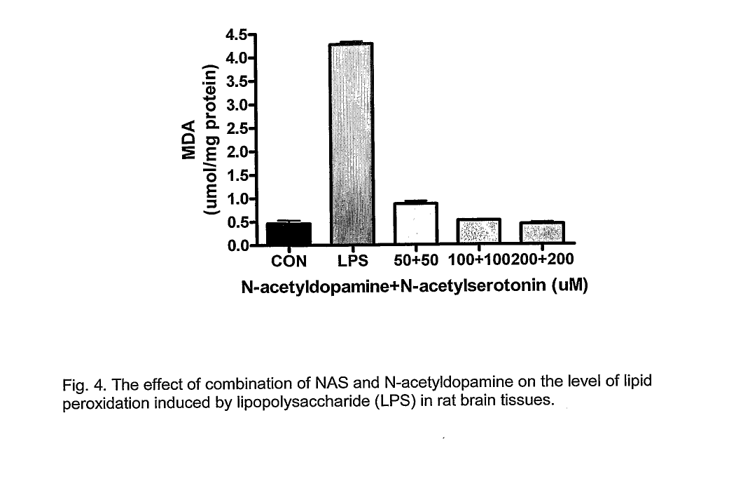

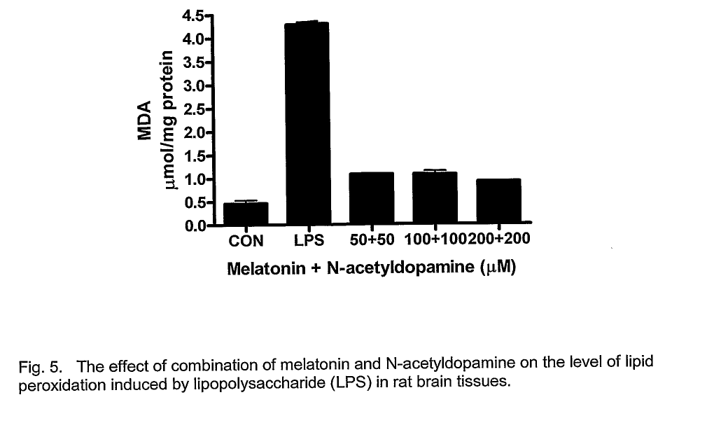

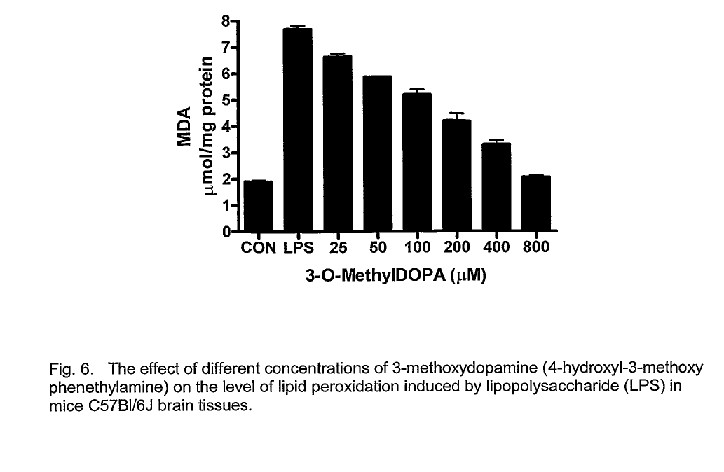

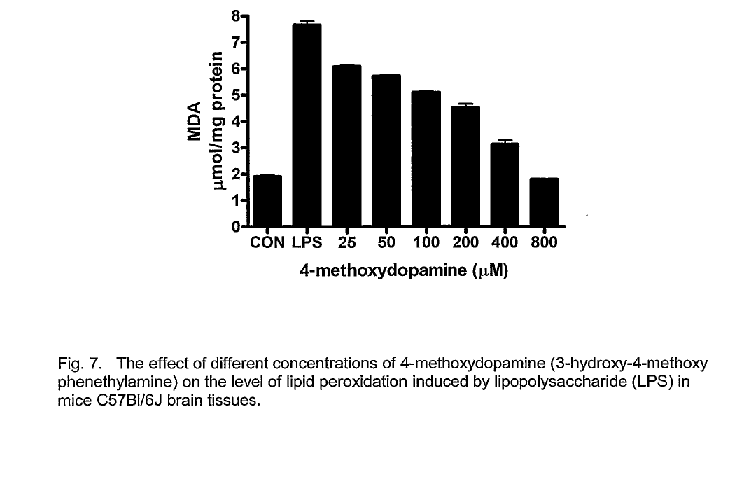

LPS induced 9-fold increase of MDA (malondialdehyde) levels in brain tissues (FIG. 1). Only lipopolysaccharide (400 μM) was added for LPS and LPS+different concentrations of N-acetyldopamine were added for all other groups. The MDA level of the LPS group was higher than in any other groups (p<0.001). N-acetyldopamine inhibited LPS-induced formation of MDA in a dose-dependent manner starting from concentration of 50 μM and reaching complete inhibition at 200 μM (FIG. 1). The effect of NAS on LPS-induced lipid peroxidation was similar to that of N-acetyldopamine (FIG. 2). Only lipopolysaccharide (400 μM) was added for LPS and LPS+different concentrations of melatonin were added for all other groups. The MDA level of the LPS group was higher than in any other groups (p<0.001). Melatonin reached statistically significant inhibition of LPS-induced lipid peroxidation at the concentration of 800 μM (FIG. 3). Only lipopolysaccharide (400 μM) was added for LPS and LPS+different concentrations of N-acetyldopamine and N-acetylserotonin were added for all other groups. The MDA level of the LPS+melatonin (800 μM) group was higher than in any other groups (p<0.001). Combination of N-acetyldopamine and NAS exerted additive effect (FIG. 4). Only lipopolysaccharide (400 μM) was added for LPS and LPS+different concentrations of N-acetyldopamine and melatonin were added for all other groups. The MDA level of LPS only group was significantly different from any other groups (p<0.001). Combination of N-acetyldopamine and melatonin did not result in additive effect (FIG. 5). Only lipopolysaccharide (400 μM) was added for LPS and LPS+different concentrations of 3-methoxydopamine (4-hydroxyl-3-methoxy phenethylamine) were added for all other groups. 3-methoxydopamine (4-hydroxyl-3-methoxy phenethylamine) inhibited LPS-induced formation of MDA in C57BI/6J mouse brain in a dose-dependent manner reaching 50% inhibition at 200μ (FIG. 6). The effect of different concentrations of 4-methoxydopamine (3-hydroxy-4-methoxy phenethylamine) on the level of lipid peroxidation induced by lipopolysaccharide (LPS) in mice C57BI/6J brain tissues was similar to that of 3-methoxydopamine (FIG. 7)

### Example 2

The effect of N-substituted dopamine derivatives on FeCl₂-induced oxidative damage was evaluated in mice C57BI/J6 brain tissues.

TBARS Determination

Brain tissues from C57BI/J6 rats were homogenized in 20 mM pH 7.4 Tris buffer. The homogenates were then incubated (1 hr) in solvent alone (control) or in solvent+FeCl₂, N-acetyldopamine+FeCl₂, N-methyldopamine (deoxyepinephrine)+FeCl₂, 3-methoxyldopamine+FeCl₂, and 4-methoxydopamine (3-hydroxy-4-methoxy phenethylamine HCl)+FeCl₂. TBARS were added, and homogenates were incubated for 20 min. in a boiling water bath. After cooling, the absorbance was measured spectrophotometrically at 532 nm. TBARS concentrations were derived from a standard curve of MDA (Oxis International, Portland, Oreg.). Protein was determined by Lowry method (Sigma Chem. Co., St. Louis, Mo.). Results are expressed as mean +S.D. (n=6). Data was treated by one-way ANOVA and Student's t-test

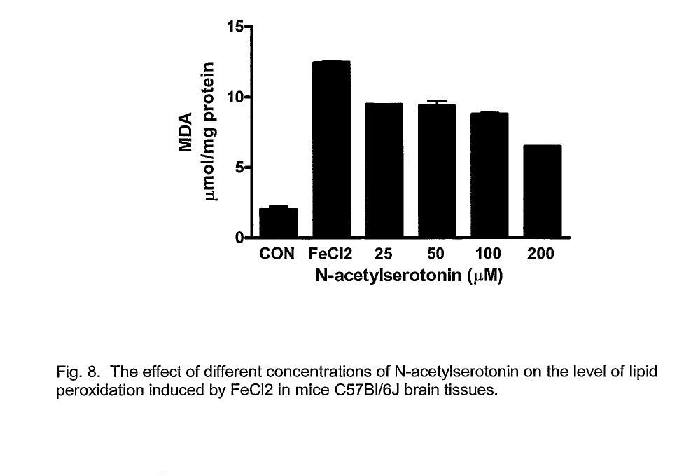

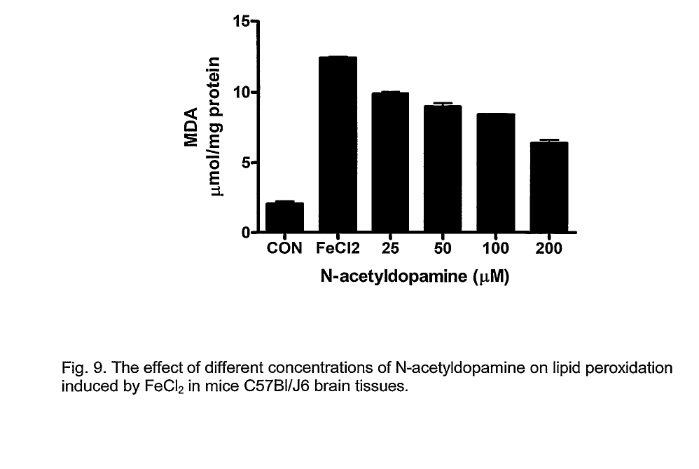

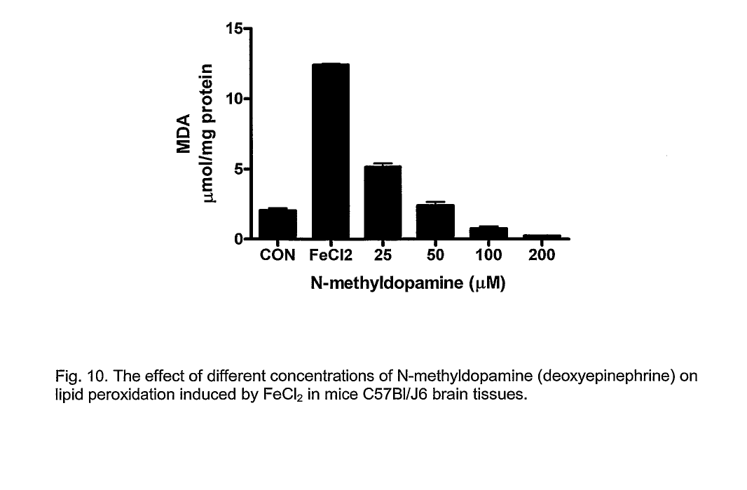

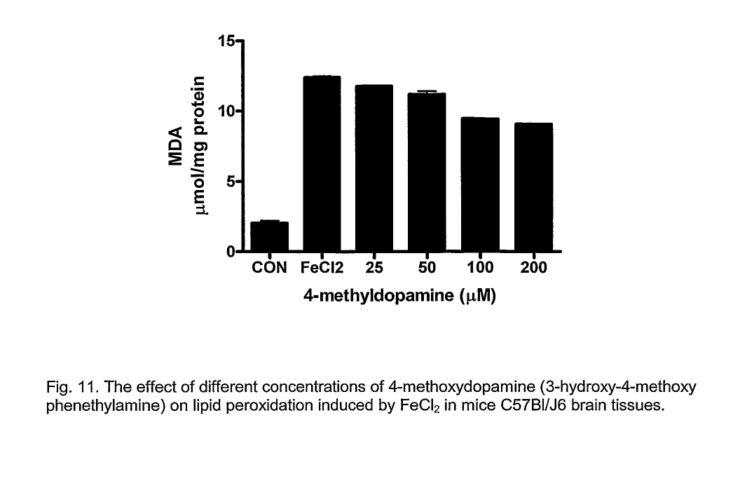

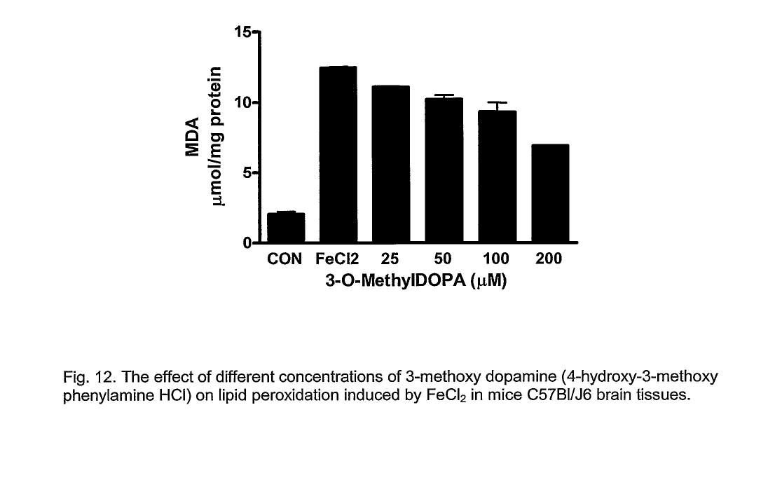

Only FeCl₂ (1 mM) was added for FeCl₂ and FeCl₂+different concentrations of N-substituted dopamine derivatives all other groups in FIGS. 8-12. FeCl₂ induced more than 5 times of MDA (malondialdehyde) levels in brain tissues (FIGS. 8-12). MDA level of FeCl2 only group was significantly different from any other groups (p<0.001). N-substituted dopamine derivatives (FIGS. 8, 10, 11 and 12) and N-acetyldopamine derivatives (FIG. 9) inhibited FeCl₂-induced formation of MDA in a dose-dependent manner. N-methyldopamine (deoxyepinephrine) exerted most prominent effect starting from concentration of 25 μM and reaching complete inhibition at 200 μM. (FIG. 10).

### REFERENCES

1. Tracey, K. J.; and A. Cerami. Tumornecrosis factor: a pleiotropic cytokine and therapeutic target. Ann. Rev. Med. 45:491-503; 1994
2. Reynolds, F. D.; Dauchy, R.; Blask, D.; Dietz, P. A.; Lynch, D.; Zuckerman, R. The pineal glandhormone melatonin improves survival in a rat model of sepsis shock induced by zymosan A. Surgery 134:474-479; 2003
3. Faure, E.; Thomas, L.; Xu, H.; Medvedev, A.; Equils, O.; Arditi, M. Bacterial lipopolysaccharide and IFN-gamma induce Toll-like receptor 2 and Toll-like receptor 4 expression in human endothelial cells: role of NF-kappa B activation. J. Immunol. 166:2018-2024; 2001.
4. Sewerynek, E.; Melchiorri, D.; Chen, L.; Reiter, R. J. Melatonin reduces both basal and bacterial lipopolysaccharide-induced lipid peroxidation in vitro. Free Radic. Biol. Med. 19:903-909; 1995
5. Maestroni, G. J. M. Melatonin as a therapeutic agent in experimental endotoxic shock J. Pineal. Res. 20: 84-89; 1996.
6. Requintina, P. J.; Oxenkrug, G. F. Differential effects of lipopolysaccharide on lipid peroxidation in F344N, SHR rats and BALB/c mice, and protection of melatonin and NAS against its toxicity. Ann. N.Y. Acad. Sci. 993:325-333; 2003
7. Gitto, E.; Romeo, C.; Reiter, R. J.; Impellizzeri, P.; Pesce, S.; Basile, M.; Antonuccio, P.; Trimarchi, G.; Gentile, C.; Barberi, I.; Zuccarello, B. Melatonin reduces oxidative stress in surgical neonates. J. Pediatr. Surg. 39:184-189; 2004
8. Young, I. M.; Leone, R. M.; Francis, P.; Stovell, P.; Silman, R. E. Melatonin is metabolized to N-acetyl serotonin and 6-hydroxymelatonin in man. J. Clin. Endocrinol. Metab. 60:114-119; 1985
9. Klemm, P.; Ostrowsk, i J.; Morath, T.; Gruber, C.; Martorana, P. A.; Henning, R. N-Acetylserotonin prevents the hypotension induced by bacterial lipopolysaccharides in the rat. Eur. J. Pharmacol. 250: R9-10; 1993
10. Sacco, S.; Aquilini, L.; Ghezzi, P.; Pinza, M.; Guglielmotti, A. Mechanism of the inhibitory effect of melatonin on tumor necrosis factor production in vivo and in vitro. Eur J Pharmacol. 343:249-255; 1998
11. Klemm, P.; Hecker, M.; Stockhausen. H.; Wu, C. C.; Thiemermann, C. Inhibition by N-acetyl-5-hydroxytryptamine of nitric oxide synthase expression in cultured cells and in the anaesthetized rat. Br. J. Pharmacol. 115:1175-1181; 1995
12. Reiter, R. J.; Tan, D. X.; Burkhardt, S. Reactive oxygen and nitrogen species and cellular and organismal decline: amelioration with melatonin. Mech. Ageing Dev. 123:1007-1019; 2002
13. Katoh, S.; Sueoka, T.; and S. Yamada. Direct inhibition of brain sepiapterine reductase by catecholamine and indoleamine. Biochem. Biophys. Res. Comm. 105:75-81; 1982
14. Smith, G. K.; Duch, D. S.; Edelstein, M. P.; E. C. Bigham, New inhibitors of sepiapterine reductase. Lack of an effect of intracellular tetrahydrobiopterine depletion upon in vito proliferation of two human cell lines. J. Biol. Chem. 267: 5599-5607; 1992
15. Thony, B.; Auerbach, G.; Blau, N. Tetrahydrobiopterin biosynthesis, regeneration and functions. Biochem. J. 347 (Pt 1): 1-16; 2000

All references cited herein are incorporated by reference.

What is claimed is:

1. A method for treating an animal or human suffering abnormal lipid peroxidation comprising: administering an effective amount of a compound of Formula (I): 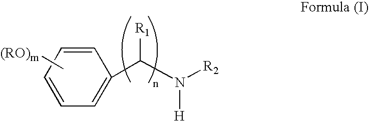
or a pharmaceutically acceptable salt or prodrug form thereof,
wherein
R is independently hydrogen, optionally substituted C₁-C₆ alkyl, optionally substituted C₂-C₆ alkenyl, and optionally substituted C₂-C₆ alkynyl;
m is 0, 1, 2, 3, 4 or 5;
R₁ is independently hydrogen, halogen, optionally substituted C₁-C₆ alkyl, optionally substituted C₂-C₆ alkenyl, optionally substituted C₂-C₆ alkynyl, optionally substituted C₁-C₆ alkoxy, optionally substituted C₁-C₆ alkylthio, optionally substituted C₁-C₆ alkylsulfinyl, optionally substituted C₁-C₆ alkylsulfonyl, optionally substituted C₁-C₆ aminoalkyl, optionally substituted carbocyclic aryl, or optionally substituted aralkyl;
n is 1, 2, or 3;
R₂ is optionally substituted C₁-C₆ alkyl, or —C(═O)R₃;
R₃ is independently optionally substituted C₁-C₆ alkyl, optionally substituted C₂-C₆ alkenyl, optionally substituted C₂-C₆ alkynyl, optionally substituted or unsubstituted carbocyclic aryl, or an optionally substituted heteroaromatic or heteroalicyclic group having from 1 to 3 rings, 3 to about 8 ring members in each ring and 1 to about 3 hetero atoms.

2. The method of claim 1, wherein;
R is independently hydrogen, or optionally substituted C₁-C₄ alkyl;
m is 1 or 2;
R₁ is each independently hydrogen, halogen, or optionally substituted C₁-C₄ alkyl;
n is 1, 2, or 3;
R₂ is —CH₃, or —C(═O)R₃;
R₃ is independently optionally substituted C₁-C₄ alkyl; optionally substituted C₂-C₆ alkenyl or optionally substituted C₃-C₄ alkynyl.

3. The method of claim 1, wherein the compound of Formula (I) is selected from the group consisting of N-acetyldopamine, N-chloroacetyldopamine, N-methyldopamine, and N-acetyl-m-tyramine.

4. The method of claim 3, wherein the compound of Formula (I) is N-acetyldopamine.

5. The method of claim 3, wherein the compound of Formula (I) is N-methyldopamine.

6. The method of claim 1, wherein the lipid peroxidation is the result of a disease or disorder.

7. The method of claim 6, wherein the disease or disorder is septic shock.

8. The method of claim 6, wherein the disease or disorder is selected from the group consisting of microbial infections, carcinogenesis, mutation, degenerative disorders, Parkinson's, Alzheimer's, multiple sclerosis, burns, aging, and the toxic effects of chemotherapy and radiation therapy.

9. A method for treating an animal or human suffering abnormal lipid peroxidation comprising: administering an effective amount of a compound of Formula (I) in combination with an effective amount of N-acetylserotonin; 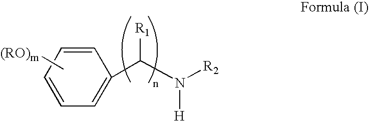
or a pharmaceutically acceptable salt or prodrug form thereof,
wherein
R is independently hydrogen, optionally substituted C₁-C₆ alkyl, optionally substituted C₂-C₆ alkenyl, and optionally substituted C₂-C₆ alkynyl;
m is 0, 1, 2, 3, 4 or 5;
R₁ is independently hydrogen, halogen, optionally substituted C₁-C₆ alkyl, optionally substituted C₂-C₆ alkenyl, optionally substituted C₂-C₆ alkynyl, optionally substituted C₁-C₆ alkoxy, optionally substituted C₁-C₆ alkylthio, optionally substituted C₁-C₆ alkylsulfinyl, optionally substituted C₁-C₆ alkylsulfonyl, optionally substituted C₁-C₆ aminoalkyl, optionally substituted carbocyclic aryl, or optionally substituted aralkyl;
n is 1, 2, or 3;
R₂ is optionally substituted C₁-C₆ alkyl, or —C(═O)R₃;
R₃ is independently optionally substituted C₁-C₆ alkyl, optionally substituted C₂-C₆ alkenyl, optionally substituted C₂-C₆ alkynyl, optionally substituted or unsubstituted carbocyclic aryl, or an optionally substituted heteroaromatic or heteroalicyclic group having from 1 to 3 rings, 3 to about 8 ring members in each ring and 1 to about 3 hetero atoms.

10. The method of claim 9, wherein;
R is independently hydrogen, or optionally substituted C₁-C₄ alkyl;
m is 1 or 2;
R₁ is each independently hydrogen, halogen, or optionally substituted C₁-C₄ alkyl;
n is 1, 2, or 3;
R₂ is —CH₃, or —C(═O)R₃;
R₃ is independently optionally substituted C₁-C₄ alkyl; optionally substituted C₂-C₆ alkenyl or optionally substituted C₃-C₄ alkynyl.

11. The method of claim 9, wherein the compound of Formula (I) is selected from the group consisting of N-acetyldopamine, N-chloroacetyldopamine, N-methyldopamine, and N-acetyl-m-tyramine.

12. The method of claim 11, wherein the compound of Formula (I) is N-acetyldopamine.

13. The method of claim 9, wherein the lipid peroxidation is the result of a disease or disorder.

14. The method of claim 13, wherein the disease or disorder is septic shock.

15. The method of claim 13, wherein the disease or disorder is selected from the group consisting of microbial infections, carcinogenesis, mutation, degenerative disorders, Parkinson's, Alzheimer's, multiple sclerosis, burns, aging, and the toxic effects of chemotherapy and radiation therapy.

16. A pharmaceutical composition for treating an animal or human suffering abnormal lipid peroxidation comprising a pharmaceutically effective amount of a compound of Formula (I); 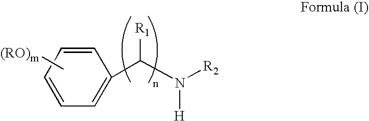
or a pharmaceutically acceptable salt or prodrug form thereof,
wherein
R is independently hydrogen, optionally substituted C₁-C₆ alkyl, optionally substituted C₂-C₆ alkenyl, and optionally substituted C₂-C₆ alkynyl;
m is 0, 1, 2, 3, 4 or 5;
R₁ is independently hydrogen, halogen, optionally substituted C₁-C₆ alkyl, optionally substituted C₂-C₆ alkenyl, optionally substituted C₂-C₆ alkynyl, optionally substituted C₁-C₆ alkoxy, optionally substituted C₁-C₆ alkylthio, optionally substituted C₁-C₆ alkylsulfinyl, optionally substituted C₁-C₆ alkylsulfonyl, optionally substituted C₁-C₆ aminoalkyl, optionally substituted carbocyclic aryl, or optionally substituted aralkyl;
n is 1, 2, or 3;
R₂ is optionally substituted C₁-C₆ alkyl, or —C(═O)R₃;
R₃ is independently optionally substituted C₁-C₆ alkyl, optionally substituted C₂-C₆ alkenyl, optionally substituted C₂-C₆ alkynyl, optionally substituted or unsubstituted carbocyclic aryl, or an optionally substituted heteroaromatic or heteroalicyclic group having from 1 to 3 rings, 3 to about 8 ring members in each ring and 1 to about 3 hetero atoms;
and a pharmaceutically acceptable carrier.

17. The pharmaceutical composition of claim 16, wherein;
R is independently hydrogen, or optionally substituted C₁-C₄ alkyl;
m is 1 or 2;
R₁ is each independently hydrogen, halogen, or optionally substituted C₁-C₄ alkyl;
n is 1, 2, or 3; R₂ is —CH₃, or —C(═O)R₃;
R₃ is independently optionally substituted C₁-C₄ alkyl; optionally substituted C₂-C₆ alkenyl or optionally substituted C₃-C₄ alkynyl.

18. The pharmaceutical composition of claim 16, wherein the compound of Formula (I) is selected from the group consisting of N-acetyldopamine, N-chloroacetyldopamine, N-methyldopamine, and N-acetyl-m-tyramine.

19. The pharmaceutical composition of claim 18, wherein the compound of Formula (I) is N-acetyldopamine.

20. The pharmaceutical composition of claim 18, wherein the compound of Formula (I) is N-methyldopamine.

21. The pharmaceutical composition of claim 18, wherein the lipid peroxidation is the result of a disease or disorder.

22. The pharmaceutical composition of claim 21, wherein the disease or disorder is septic shock.

23. The pharmaceutical composition of claim 21, wherein the disease or disorder is selected from the group consisting of microbial infections, carcinogenesis, mutation, degenerative disorders, Parkinson's, Alzheimer's, multiple sclerosis, burns, aging, and the toxic effects of chemotherapy and radiation therapy.

24. A pharmaceutical composition for treating an animal or human suffering abnormal lipid peroxidation comprising a pharmaceutically effective amount of a compound of Formula (I) in combination with an effective amount of N-acetylserotonin; 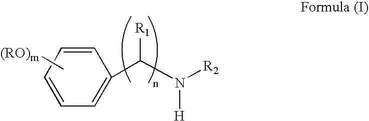
or a pharmaceutically acceptable salt or prodrug form thereof,
wherein
R is independently hydrogen, optionally substituted C₁-C₆ alkyl, optionally substituted C₂-C₆ alkenyl, and optionally substituted C₂-C₆ alkynyl;
m is 0, 1, 2, 3, 4 or 5;
R₁ is independently hydrogen, halogen, optionally substituted C₁-C₆ alkyl, optionally substituted C₂-C₆ alkenyl, optionally substituted C₂-C₆ alkynyl, optionally substituted C₁-C₆ alkoxy, optionally substituted C₁-C₆ alkylthio, optionally substituted C₁-C₆ alkylsulfinyl, optionally substituted C₁-C₆ alkylsulfonyl, optionally substituted C₁-C₆ aminoalkyl, optionally substituted carbocyclic aryl, or optionally substituted aralkyl;
n is 1, 2, or 3;
R₂ is optionally substituted C₁-C₆ alkyl, or —C(═O)R₃;
R₃ is independently optionally substituted C₁-C₆ alkyl, optionally substituted C₂-C₆ alkenyl, optionally substituted C₂-C₆ alkynyl, optionally substituted or unsubstituted carbocyclic aryl, or an optionally substituted heteroaromatic or heteroalicyclic group having from 1 to 3 rings, 3 to about 8 ring members in each ring and 1 to about 3 hetero atoms;
and a pharmaceutically acceptable carrier.

25. The pharmaceutical composition of claim 24, wherein;
R is independently hydrogen, or optionally substituted C₁-C₄ alkyl;
m is 1 or 2;
R₁ is each independently hydrogen, halogen, or optionally substituted C₁-C₄ alkyl;
n is 1, 2, or 3;
R₂ is —CH₃, or —C(═O)R₃;
R₃ is independently optionally substituted C₁-C₄ alkyl; optionally substituted C₂-C₆ alkenyl or optionally substituted C₃-C₄ alkynyl.

26. The pharmaceutical composition of claim 24, wherein the compound of Formula (I) is selected from the group consisting of N-acetyldopamine, N-chloroacetyldopamine, N-methyldopamine, and N-acetyl-m-tyramine.

27. The pharmaceutical composition of claim 26, wherein the compound of Formula (I) is N-acetyldopamine.

28. The pharmaceutical composition of claim 26, wherein the compound of Formula (I) is N-methyldopamine.

29. The pharmaceutical composition of claim 24, wherein the lipid peroxidation is the result of a disease or disorder.

30. The pharmaceutical composition of claim 29, wherein the disease or disorder is septic shock.

31. The pharmaceutical composition of claim 29, wherein the disease or disorder is selected from the group consisting of microbial infections, carcinogenesis, mutation, degenerative disorders, Parkinson's, Alzheimer's, multiple sclerosis, burns, aging, and the toxic effects of chemotherapy and radiation therapy.
